(19) United States
(12) Patent Application Publication
(10) Pub. No.: US 2026/0038657 A1
(43) Pub. Date: Feb. 5, 2026

(54) INFORMATION PROCESSING DEVICE, INFORMATION PROCESSING METHOD, AND STORAGE MEDIUM

(71) Applicant: JVCKENWOOD Corporation, Yokohama-shi (JP)

(72) Inventor: Takamitsu Shimakura, Yokohama-shi (JP)

(21) Appl. No.: 19/318,417

(22) Filed: Sep. 4, 2025

Related U.S. Application Data
(63) Continuation of application No. PCT/JP2024/006214, filed on Feb. 21, 2024.

(30) Foreign Application Priority Data
Mar. 13, 2023 (JP) 2023-038953

(51) Int. Cl. G16H 10/60

(52) U.S. Cl. CPC G16H 10/60

(57) ABSTRACT

An information processing device includes a brain information obtaining unit that obtains the brain information of a subject; an output unit that outputs the brain information, which is obtained by the brain information obtaining unit, to the subject; and a delaying unit that delays, by a predetermined period of time set in advance, the timing at which the output unit outputs the brain information to the subject.

### CROSS-REFERENCE TO RELATED APPLICATIONS

This application is a Continuation of PCT International Application No. PCT/JP2024/006214 filed on Feb. 21, 2024 which claims the benefit of priority from Japanese Patent Application No. 2023-038953, filed on Mar. 13, 2023, the entire contents of both of which are incorporated herein by reference.

### BACKGROUND OF THE INVENTION

### 1. Field of the Invention

The application concerned is related to an information processing device, an information processing method, and a computer program product.

### 2. Description of the Related Art

In recent years, there have been advancements in the technology for measuring brain activation information; and the technology of a brain-machine interface, which serves as an interface between the brain and the outside, is becoming feasible. In Japanese Patent Application Laid-open No. 2015-205183 mentioned below, the explanation is given about deciding on the target frequency corresponding to the brain state; measuring the electrical output of the brain of the user as received from a sensor coupled to the user; deciding on the present frequency associated to the brain of the user based on the electrical output; and deciding on the difference between the present frequency and the target frequency.

However, in Japanese Patent Application Laid-open No. 2015-205183 mentioned above, although it is possible to distinctively determine the cerebrum state, there is no disclosure about a method for controlling the brain wave signals without having to use complex transfer functions.

### SUMMARY OF THE INVENTION

It is an object of the present invention to at least partially solve the problems in the conventional technology.

The above and other objects, features, advantages and technical and industrial significance of this invention will be better understood by reading the following detailed description of presently preferred embodiments of the invention, when considered in connection with the accompanying drawings.

An information processing device according to the present disclosure comprising: a brain information obtaining unit that obtains brain information of a subject; an output unit that outputs the brain information, which is obtained by the brain information obtaining unit, to the subject; and a delaying unit that delays, by a predetermined period of time set in advance, timing at which the output unit outputs the brain information to the subject.

An information processing method according to the present disclosure comprising: obtaining brain information of a subject; storing the obtained brain information; and
delaying, by a predetermined period of time set in advance, timing of outputting the stored brain information to the subject.

A non-transitory computer-readable storage medium storing a program causing a computer according to the present disclosure to execute: obtaining brain information of a subject; storing the obtained brain information; and delaying, by a predetermined period of time set in advance, timing of outputting the stored brain information to the subject.

### BRIEF DESCRIPTION OF THE DRAWINGS

FIG. 1 is a block configuration diagram illustrating an information processing device according to an embodiment; and

FIG. 2 is a flowchart for explaining an information processing method according to the present embodiment.

### DETAILED DESCRIPTION OF THE PREFERRED EMBODIMENTS

An exemplary embodiment of an information processing device, an information processing method, and a computer program product according to the application concerned is described below in detail with reference to the accompanying drawings. However, the application concerned is not limited by the embodiment described below.

### Information Processing Device

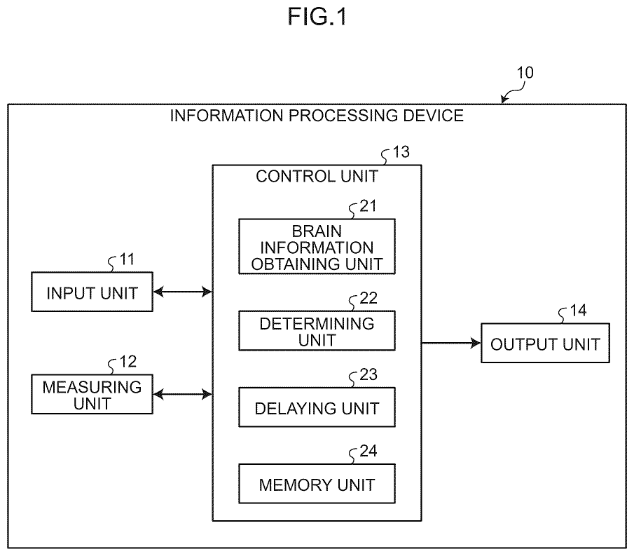

FIG. 1 is a block configuration diagram illustrating an information processing device according to the present embodiment.

In an information processing device 10 illustrated in FIG. 1, the electrical signals of the brain waves, which represent the brain information, obtained from a subject are fed back (output) to the subject only after a delay; and hence the brain activity of the subject is held down.

It is believed that the brain of a person constantly monitors the prediction errors occurring between the autonomous nervous system inside the body and the external motor control system, and at the same time performs the task of giving feedback to the self-expected state and making a prediction in an internal model. The feedback to the brain (particularly, the sensory organs and the motor control) is of considerable importance. Hence, if an error occurs in giving feedback to the brain, it is believed that complex processing becomes necessary in the motor system and the autonomous nervous system.

Moreover, there is a method by which feedbacking is utilized with respect to a person who is talking, so that the utterance of that person is forcibly obstructed. In that method, a delay of about a few hundred seconds is applied to the utterance and a sound is fed back to the auditory sense of the speaker. That results in obstruction of the normal utterance of the speaker. The delay period corresponds to the period of time of reaction of the phenomena between the sensory organs and the brain; and the talking gets obstructed due to that action. Alternatively, when a person is singing and when the voice of that person is fed back with some delay, then the pitch of the song goes off thereby affecting the brain in a significant manner.

As explained above, in the information processing device 10, the electrical signals of the brain waves obtained from a user (a subject) are fed back to that user only after delay, and hence the brain activity of the user is held down. The information processing device 10 includes an input unit 11, a measuring unit 12, a control unit 13, and an output unit 14.

The input unit 11 is connected to the control unit 13. The input unit 11 is configured to be operable by a user and is capable of inputting various signals to the control unit 13. For example, to the control unit 13, the input unit 11 inputs a start signal for starting to give feedback of the brain waves to the user, and inputs an end signal for ending the feedback of the brain waves to the user. The input unit 11 can be implemented using, for example, a touch-sensitive panel, a button, a switch, or a keyboard.

The measuring unit 12 is connected to the control unit 13. The control unit 13 applies a measurement signal to the measuring unit 12 based on a computer program. Then, based on the measurement signal input from the control unit 13, the measuring unit 12 measures the brain information of the user.

The measuring unit 12 is a sensor that detects the brain information of the user. The measuring unit 12 obtains, for example, the electrical signals of the brain waves as the brain information. As the measuring unit 12, for example, it is possible to use a measurement device including invasive electrodes.

The control unit 13 includes a brain information obtaining unit 21, a determining unit 22, a delaying unit 23, and a memory unit 24. The control unit 13 is configured using, for example, an arithmetic circuit such as a central processing unit (CPU).

The brain information obtaining unit 21 is connected to the measuring unit 12. The brain information obtaining unit 21 controls the measuring unit 12 and causes the measuring unit 12 to detect the brain information of the user. Then, the brain information obtaining unit 21 obtains the brain information of the user as measured by the measuring unit 12.

The brain information obtaining unit 21 is connected to the determining unit 22. Based on the electrical signals of the brain waves of the user as obtained by the brain information obtaining unit 21, the determining unit 22 determines whether or not to provide the user with the feedback of the electrical signals of the brain waves of that user. For example, based on the degree of activity (the degree of inactivity) and the degree of comfort (the degree of discomfort) indicating the brain state of the user, the determining unit 22 determines whether or not to perform feedback processing. More particularly, when the degree of activity (the degree of inactivity) or the degree of comfort (the degree of discomfort) is lower than a threshold value set in advance, the determining unit 22 instructs implementation of feedback processing. On the other hand, when the degree of activity (the degree of inactivity) or the degree of comfort (the degree of discomfort) is higher than the threshold value set in advance, the determining unit 22 does not instruct implementation of feedback processing.

In order to determine the degree of activity (the degree of inactivity) or the degree of comfort (the degree of discomfort) representing the brain state of the user, it is possible to implement various technologies. For example, from among the electrical signals of the brain waves of the user as obtained by the brain information obtaining unit 21, the determining unit 22 can measure the response of the 0 bandwidth in the region of interest in the brain (i.e., can measure the electrical signals of an EEG) and calculate the degree of comfort (the degree of discomfort). Alternatively, from among the electrical signals of the brain waves of the user as obtained by the brain information obtaining unit 21, the determining unit 22 can measure the response of the B bandwidth of the region of interest in the brain (i.e., can measure the electrical signals of an EEG) and calculate the degree of activity (the degree of inactivity).

It is desirable that the threshold values for the degree of activity (the degree of inactivity) and the degree of comfort (the degree of discomfort) are set on a user-by-user basis. In that case, a plurality of relationships of the emotions of a user with the degree of activity (the degree of inactivity) and the degree of comfort (the degree of discomfort) is obtained in advance. Then, among the plurality of obtained relationships of a user with the degree of activity (the degree of inactivity) and the degree of comfort (the degree of discomfort), it is desirable that the threshold values are held according to the emotions of the user indicating activeness or comfort.

The determining unit 22 is connected to the delaying unit 23. According to the determination result obtained by the determining unit 22, the delaying unit 23 delays the timing of outputting the electrical signals of the brain waves (i.e., the brain information) to the output unit 14. Thus, when the determining unit 22 determines that the degree of activity or the degree of comfort representing the brain state of the user is low, that is, when the determining unit 22 determines that the brain of the user is not activated or that the user is feeling discomfort, the delaying unit 23 outputs a delay instruction to the output unit 14 for delaying, by a predetermined period of time set in advance, the timing of outputting the electrical signals of the brain waves (i.e., the brain information) to the user. At that time, the delaying unit 23 sets the predetermined period of time, by which the output unit 14 delays the electrical signals of the brain waves, to be in the range between 200 msec and 500 msec. On the other hand, when the determining unit 22 determines that the degree of activity or the degree of comfort representing the brain state of the user is high, that is, when the determining unit 22 determines that the brain of the user is activated or that the user is not feeling discomfort, the delaying unit 23 does not output a delay instruction to the output unit 14.

The memory unit 24 is connected to the brain information obtaining unit 21. The memory unit 24 stores therein the electrical signals of the brain waves (i.e., the brain information) of a user as obtained by the brain information obtaining unit 21. Moreover, the memory unit 24 stores therein various threshold values that the control unit 13 uses while performing information processing. Furthermore, the memory unit 24 stores therein a computer program that the control unit 13 executes to perform information processing. The memory unit 24 is an external storage device such as a hard disk drive (HDD), or is a memory.

The control unit 13 is connected to the output unit 14. The output unit 14 outputs the control result, which is obtained by the control unit 13, to the user. That is, the output unit 14 is able to provide the user with the feedback of the electrical signals of the brain waves of that user as obtained by the brain information obtaining unit 21 and stored in the memory unit 24. At that time, the output unit 14 outputs the electrical signals of the brain waves (i.e., the brain information) to the user based on the determination result obtained by the determining unit 22. Moreover, at the time of outputting the electrical signals of the brain waves (i.e., the brain information) to the user, the output unit delays the output of the electrical signals of the brain waves (i.e., the brain information) to the user based on a command signal received from the delaying unit 23.

That is, upon determining that the degree of activity or the degree of comfort representing the brain state of the user is low, the determining unit 22 outputs that determination result to the delaying unit 23. When the determination result indicating that the degree of activity or the degree of comfort representing the brain state of the user is low is received from the determining unit 22, the delaying unit 23 instructs the output unit 14 to delay, by a predetermined period of time, the feedback of the electrical signals of the brain waves, which are stored in the memory unit 24, to the brain of that user.

The output unit 14 outputs the brain information, such as the electrical signals of the brain waves, to the user and thus stimulates the brain. Upon receiving a command from the delaying unit 23, the output unit 14 delays, by a predetermined period of time, the feedback of the electrical signals of the brain waves, which are stored in the memory unit 24, to the brain of that user. As the output unit 14, for example, it is possible to use an output device including invasive electrodes. Meanwhile, the output unit 14 can be configured in an integrated manner with the measuring unit 12.

### Information Processing Method

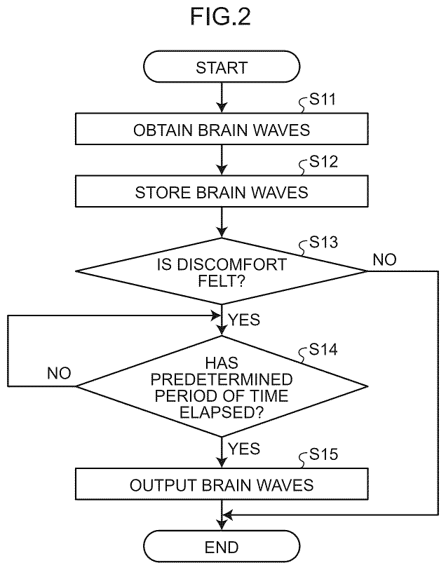

FIG. 2 is a flowchart for explaining an information processing method according to the present embodiment. Herein, every time the end is reached, the information processing method is repeated after a predetermined sampling period.

As illustrated in FIGS. 1 and 2, at Step S11, from the measuring unit 12 and within a brain wave reading period shorter than a predetermined period of time representing the delay period, the brain information obtaining unit 21 obtains the electrical signals of the brain waves representing the brain information from the brain cells of the user. At Step S12, the memory unit 24 stores therein the electrical signals of the brain waves as obtained by the brain information obtaining unit 21. At Step S13, the determining unit 22 determines whether or not the degree of comfort (the degree of discomfort) representing the brain state of the user is lower than a threshold value set in advance, that is, determines whether or not the user is feeling discomfort. When the determining unit 22 determines that the degree of comfort (the degree of discomfort) representing the brain state of the user is not lower than the threshold value set in advance, that is, determines that the user is not feeling discomfort (No), the system control exits the routine.

On the other hand, when the determining unit 22 determines that the degree of comfort (the degree of discomfort) representing the brain state of the user is lower than the threshold value set in advance, that is, determines that the user is feeling discomfort (Yes), the system control proceeds to Step S14. At Step S14, when the determination result indicating that the user has a low degree of comfort, that is, the user is feeling discomfort is input from the determining unit 22, the delaying unit 23 determines whether or not a predetermined period of time has elapsed since the brain information obtaining unit 21 obtained the electrical signals of the brain waves. Herein, the predetermined period of time represents the delay period for delaying the feedback of the electrical signals of the brain waves of the user to the brain of the user.

When it is determined that the predetermined period of time has not elapsed (No), the delaying unit 23 maintains the current state. On the other hand, when it is determined that the predetermined period of time has elapsed (Yes), the system control proceeds to Step $15. At Step S15, after the elapse of the predetermined period of time, the delaying unit 23 instructs the output unit 14 to delay, by a predetermined period of time, the feedback of the electrical signals of the brain waves of the user, which are stored in the memory unit 24, to the brain of that user. Upon receiving the command from the delaying unit 23, the output unit 14 delays, by a predetermined period of time, the feedback of the electrical signals of the brain waves of the user, which are stored in the memory unit 24, to the brain of that user. As a result, the brain wave signals of the user are controlled and the thinking stops.

The sensory organs of a person are said to have the average response rate of 68 msec at 20 years of age, 86 msec at 40 years of age, and 106 msec at 60 years of age. Moreover, when a person receives signals from a sensory organ and the brain makes a decision by assuming various aspects, the time taken midway for consecutive processing of commands is said to be between 150 msec to 300 msec. In that view, with respect to adults spanning from 20 years to 60 years, it is desirable that the predetermined period of time is set from about 200 msec to about 500 msec.

Given below is the specific explanation about the information processing method implemented by the information processing device 10 according to the present embodiment. Firstly, with respect to a specific action, the electrical signals of the corresponding neuron activity in the somatosensory area are recorded. Then, the same electrical signals are output to the user using an electrical input device of the invasive type, so that the tactile sense can be reproduced. As a result of applying that theory, the brain information obtaining unit 21 obtains the electrical signals of the brain waves (i.e., the electrical signals of the neuron activity) of the user; the memory unit 24 stores therein the electrical signals of the brain waves; the delaying unit 23 secures a predetermined delay period; and the output unit 14 delays, by the predetermined delay period, the feedback of the electrical signals of the brain waves to the brain of the user. As a result, the awareness of the sensory organs of the user toward the tactile sense gets obstructed, thereby making the user unable to get a good feel for the touch.

Given below is the explanation of an application example of the specific example. The brain information obtaining unit 21 obtains the electrical signals of the brain waves of the user as measured by the measuring unit 12. At that time, the brain information obtaining unit 21 performs specific classification with respect to the electrical signals of the brain waves. The electrical signals of the brain waves are evaluated as comfortable or uncomfortable in the way of “real-time evaluation of sensibility and cognitive ability using brain waves”. When the electrical signals of the brain waves are classified to be of the neuron activity corresponding to the tactile sense when the user feels uncomfortable, the feedback of the electrical signals of the brain waves to the brain of the user is delayed by a predetermined period of time. It results in obstructing the awareness of the sensory organs of the user against an uncomfortable tactile sense.

In the explanation given above, the information processing device 10 determines the comfort or discomfort of the brain waves of the user in real time. However, that is not the only possible method. Alternatively, the electrical signals of the neuron activity caused by specific tactile senses can be recorded in advance along with the corresponding determination results about comfort and discomfort, and the determination can be performed using only the electrical signals of the neuron activity caused by the tactile senses.

Meanwhile, the information processing device 10 can determine the comfort and the discomfort of the brain waves of the user in a biaxial manner of activation and nonactivation of the brain waves of the user. Meanwhile, the tactile organs of the user to be used in determination can be led by the sense of hearing, and other sensory organs can also be used.

More particularly, in the following explanation, Step S13 illustrated in the flowchart in FIG. 2 is modified; and, other than the modified step, the steps are identical to FIG. 2. At Step S13, the determining unit 22 determines whether or not the degree of comfort (the degree of discomfort) representing the brain state of the user is lower than a first threshold value set in advance, and determines whether or not the activation (nonactivation) representing the brain state of the user is higher than a threshold value set in advance. That is, the determining unit 22 determines whether or not the user is feeling discomfort and whether or not the brain of the user is activated. When the determining unit 22 determines that the degree of comfort (the degree of discomfort) representing the brain state of the user is not lower than the threshold value set in advance or determines that the activation (deactivation) representing the brain state of the user is not higher than the threshold value set in advance, that is, when the determining unit 22 determines that the user is not feeling discomfort or determines that the brain of the user is not activated (No), the system control exits the routine. On the other hand, at Step S13, when the determining unit 22 determines that the degree of comfort (the degree of discomfort) representing the brain state of the user is lower than the first threshold value set in advance and determines that the activation (deactivation) representing the brain state of the user is higher than the threshold value set in advance, the system control proceeds to Step S14. That is, when the determining unit 22 determines that the user is feeling discomfort and that the brain of the user is activated (Yes), the system control proceeds to Step S14.

When the determination is affirmative (Yes) at Step S13 that represents the modification in FIG. 2, it is determined that the user is feeling discomfort and that the brain of the user is activated. However, that is not the only possible case. Alternatively, when the determination is affirmative (Yes) at Step S13, it is possible to have any combination from among the following: the user is feeling at ease, the user is feeling discomfort, the brain of the user is activated, and the brain of the user is not activated.

Meanwhile, while the determination about the elapse of the predetermined period of time is underway at Step S14 illustrated in FIG. 2, it is also possible to separately implement the information processing method. As a result, while waiting for the elapse of the predetermined period of time, it becomes possible to prevent the stoppage in the implementation of the information processing method. Moreover, it is explained earlier that every time the end is reached, the information processing method is repeated after a predetermined sampling period. However, that is not the only possible case. Alternatively, even when the end is not yet reached, the information processing method can be repeated after a predetermined sampling period. Thus, the sequence of the information processing method can be initiated in separate threads.

In the explanation given above, the memory unit 24 is used. Instead, the brain wave reading period during which the measuring unit 12 performs reading from the brain cells can be set to be a fraction of the delay period representing a predetermined period of time, Accordingly, without involving the memory unit 24, the output unit 14 can receive a command signal from the delaying unit 23 and can directly give feedback of the electrical signals of the brain waves to the user. Moreover, it is explained that the brain information obtaining unit 21 obtains the brain waves from the brain cells and gives feedback of the brain waves to the brain cells. Alternatively, the brain information obtaining unit 21 can obtain the brain waves from the scalp and gives feedback of the brain waves to the scalp.

### Effects of Embodiment

The information processing device according to the present embodiment includes: the brain information obtaining unit 21 that obtains the brain information of a user (a subject); the output unit 14 that outputs the brain information, which is obtained by the brain information obtaining unit 21, to the user; and the delaying unit 23 that delays, by a predetermined period of time set in advance, the timing at which the output unit 14 outputs the brain information to the user.

As a result, since the current thinking is input to the subject after a delay of a predetermined period of time set in advance, the brain activity can be controlled without having to use complex transfer functions.

In the information processing device according to the present embodiment, the predetermined period of time by which the delaying unit 23 delays the output of the brain information is set to be between 200 msec and 500 msec. As a result, since the thinking is input to the subject after a delay of an appropriate period of time, the brain activity can be controlled in an effective manner.

The information processing device according to the present embodiment includes the memory unit 24 that stores therein the brain information obtained by the brain information obtaining unit 21; and the delaying unit 23 delays the output of the brain information, which is stored in the memory unit 24, by a predetermined period of time. As a result, the brain information obtained by the brain information obtaining unit 21 can be output to the user in an accurate manner.

Till now, the explanation was given about the information processing device according to the application concerned. However, the application concerned can be implemented according to various other forms other than the embodiment described above.

The constituent elements of the information processing device illustrated in the drawings are merely conceptual, and need not be physically configured as illustrated. The constituent elements, as a whole or in part, can be separated or integrated either functionally or physically based on various types of loads or use conditions.

The information processing device is configured using, for example, a computer program that is loaded as software in a memory. In the embodiment described above, the configuration is explained with reference to function blocks implemented as a result of coordination between hardware and software. Such function blocks can be implemented in various ways, such as using only hardware, or using only software, or using a combination of hardware and software.

According to the application concerned, it becomes possible to control the brain activity without having to use complex transfer functions.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An information processing device comprising:
a brain information obtaining unit that obtains brain information of a subject;
an output unit that outputs the brain information, which is obtained by the brain information obtaining unit, to the subject; and
a delaying unit that delays, by a predetermined period of time set in advance, timing at which the output unit outputs the brain information to the subject.

2. The information processing device according to claim 1, wherein the predetermined period of time by which the delaying unit delays output of the brain information is set between 200 msec and 500 msec.

3. The information processing device according to claim 1, further comprising a memory unit that stores therein the brain information obtained by the brain information obtaining unit, wherein
the delaying unit delays output of the brain information, which is stored in the memory unit, by the predetermined period of time.

4. An information processing method comprising:
obtaining brain information of a subject;
storing the obtained brain information; and
delaying, by a predetermined period of time set in advance, timing of outputting the stored brain information to the subject.

5. A non-transitory computer-readable storage medium storing a program causing a computer to execute:
obtaining brain information of a subject;
storing the obtained brain information; and
delaying, by a predetermined period of time set in advance, timing of outputting the stored brain information to the subject.
